(19) United States
(12) Patent Application Publication
(10) Pub. No.: US 2015/0065926 A1
(43) Pub. Date: Mar. 5, 2015

(54) CAPSULE-TYPE MEDICAL DEVICE AND SYSTEM

(71) Applicant: Sony Corporation, Tokyo (JP)

(72) Inventors: Takatoshi Nakamura, Kanagawa (JP); Yoichiro Sako, Tokyo (JP); Akira Tange, Tokyo (JP); Katsumi Ando, Tokyo (JP); Yuki Koga, Tokyo (JP)

(73) Assignee: Sony Corporation, Tokyo (JP)

(21) Appl. No.: 14/390,882

(22) PCT Filed: Feb. 25, 2013
(86) PCT No.: PCT/JP2013/054775

§ 371 Date: Oct. 6, 2014

(30) Foreign Application Priority Data
Apr. 12, 2012 (JP) 2012-090881

(51) Int. Cl. A61H 23/00; A61B 19/00; A61N 1/372; A61N 1/36

(52) U.S. Cl. CPC A61H 23/00; A61N 1/36167; A61B 19/52; A61N 1/37217; A61N 1/3606
USPC 601/46; 607/116; 607/59; 607/2; 607/60

(57) ABSTRACT

There is provided a capsule-type medical device including a stimulation unit configured to apply a stimulus to a predetermined site in a body cavity.

### TECHNICAL FIELD

The present disclosure relates to a capsule-type medical device and a system.

### BACKGROUND ART

In recent years, a capsule-type medical device to be put into the body of a subject has been known. As such capsule-type medical devices, a device for photographing each part in the body at random, a device for obtaining a sample and the like from inside the body, a device for discharging a medicine as disclosed in Patent Literatures 1 and 2 below and the like are known.

One of accidents that can occur at home involving a child is swallowing of a foreign substance by mistake. Particularly, cigarettes, pharmaceutical products, quasi-pharmaceutical products, battery cells and the like can be swallowed by mistake in many cases. In such a case, emergency measures such as making a child spew out the foreign substance, making a child drink water or milk and then, spew out, making a child drink water, or taking a child to hospital as soon as possible without forcing the child to spew out are taken depending on the type of object swallowed by mistake.

More specifically, if the foreign substance is to be spewed, an area deep in a throat is stimulated by a finger or a spoon so as to induce pharyngeal reflex.

### CITATION LIST

### Patent Literature

Patent Literature 1: JP 2003-325438A
Patent Literature 2: JP 2005-334331A

### SUMMARY OF INVENTION

### Technical Problem

However, the treatment of putting a finger into the throat is not hygienic, and there is a concern that the finger might damage the throat. Moreover, if the throat is to be stimulated by using a utensil such as a spoon, there is a concern that movement of the head part caused by spewing might damage the throat. Furthermore, many people are mentally reluctant to the treatment of stimulating the throat by a finger or a spoon, and the treatment is not casual as compared with a treatment of taking a medicine. In the above-described Patent Literatures 1 and 2, nothing for inducing pharyngeal reflex is disclosed.

Moreover, other than the children's accident of swallowing by mistake, when a person feels bad due to excessive drinking of alcohols, the treatment of putting a finger into the throat for spewing contents of the stomach has been performed in some cases.

On the other hand, if a foreign substance swallowed by mistake enters the stomach, natural discharge into excrement is awaited in some cases. In this case, it is effective to stimulate the colon from outside the body by massage so as to promote peristaltic movement.

As described above, the treatment of inducing the pharyngeal reflex for spewing or the treatment of discharging the foreign substance in the intestines is performed in a household on a daily basis in general, but a safe and casual method has not been particularly proposed.

Thus, in the present disclosure, a capsule-type medical device and a system which can discharge contents from inside the body are proposed.

### Solution to Problem

According to the present disclosure, there is provided a capsule-type medical device including a stimulation unit configured to apply a stimulus to a predetermined site in a body cavity.

According to the present disclosure, there is provided a system including a capsule-type medical device which has a stimulation unit configured to apply a stimulus to a predetermined site in a body cavity, and an external device configured to transmit a control signal for controlling start or end of the stimulation. The capsule-type medical device further has a receiving unit configured to receive the control signal from the external device. The stimulation unit starts or ends the stimulation to the predetermined site in accordance with the control signal received from the external device.

### Advantageous Effects of Invention

According to the present disclosure as described above, contents can be discharged from inside the body.

### BRIEF DESCRIPTION OF DRAWINGS

FIG. 1 is a view for explaining an outline of a first embodiment of the present disclosure.

FIG. 2 is a block diagram illustrating a configuration of a capsule-type medical device according to the first embodiment.

FIG. 3 is a block diagram illustrating a configuration of a capsule-type medical device according to a variation of the first embodiment.

FIG. 4 is an explanatory view illustrating the capsule-type medical device which applies a stimulus to a predetermined site by deformation.

FIG. 5 is a flowchart illustrating operation processing according to the first embodiment.

FIG. 6 is a view for explaining induction of pharyngeal reflex by stimulation of a capsule.

FIG. 7 is a block diagram illustrating a configuration of a capsule-type medical device according to a second embodiment.

FIG. 8 is a flowchart illustrating operation processing according to the second embodiment.

FIG. 9 is a view for explaining an outline of a medical system according to a third embodiment.

FIG. 10 is a block diagram illustrating a configuration of a capsule-type medical device according to the third embodiment.

FIG. 11 is a block diagram illustrating a configuration of a control device 20 according to the third embodiment.

FIG. 12 is a flowchart illustrating operation processing according to the third embodiment.

### DESCRIPTION OF EMBODIMENTS

Hereinafter, preferred embodiments of the present disclosure will be described in detail with reference to the appended drawings. Note that, in this specification and the drawings, elements that have substantially the same function and structure are denoted with the same reference signs, and repeated explanation is omitted.

Moreover, the explanation will be made in the following order:

1. First embodiment

1-1. Outline

1-2. Configuration of capsule

1-3. Operation processing

2. Second embodiment

2-1. Configuration of capsule

2-2. Operation processing

3. Third embodiment

### 4. Conclusion

### 1. FIRST EMBODIMENT

(1-1. Outline)

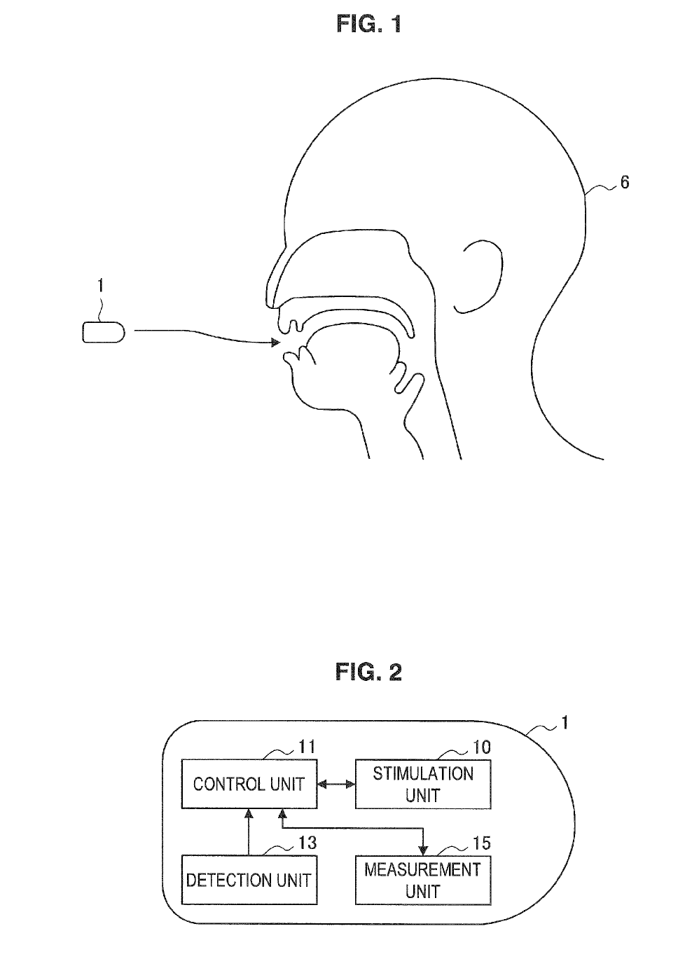

First, an outline of a first embodiment according to the present disclosure will be explained by referring to FIG. 1. As illustrated in FIG. 1, a capsule-type medical device 1 (hereinafter referred to also as a capsule 1) according to this embodiment can promote discharge of contents by being swallowed by a subject 6 and by stimulating a predetermined site in the body.

As described above, a treatment of inducing pharyngeal reflex for spewing is performed at home on a daily basis in general, but the treatment of putting a finger or a utensil into the throat is not hygienic, and there is a concern that the throat might be damaged.

Thus, according to this embodiment, there is provided a capsule-type medical device which can let the contents in the body be discharged safely and casually by means of an act with less resistance such as taking a medicine.

An outline of this embodiment is explained as above. Subsequently, a configuration and operation processing of the capsule 1 according to this embodiment will be explained in order.

(1-2. Configuration of Capsule)

FIG. 2 is a block diagram illustrating the configuration of the capsule 1 according to the first embodiment. As illustrated in FIG. 2, the capsule 1 has a stimulation unit 10, a control unit 11, a detection unit 13, and a measurement unit 15.

The detection unit 13 has a function of detecting a trigger for starting the operation processing. For example, the detection unit 13 may detect an operation of a power switch (not shown) as a trigger or may detect application of a pressure on the entire capsule 1 from outside as a trigger. If the detection unit 13 is to detect the application of a pressure from outside as a trigger, the subject 6 can also start the operation processing by biting the capsule 1 in the mouth or swallowing the capsule 1.

The control unit 11 controls each configuration of the capsule 1. Specifically, when the detection unit 13 detects the trigger, the control unit 11 applies a stimulus to a predetermined site in the body cavity by controlling the stimulation unit 10.

Moreover, when the detection unit 13 detects the trigger, the control unit 11 controls the measurement unit 15 so as to start counting and may apply a stimulus to the predetermined site in the body cavity by controlling the stimulation unit 10 when preset time is counted. For example, the control unit 11 can apply a stimulus at timing when the capsule 1 reaches the root of the tongue or the pharynx if the subject 6 swallows the capsule 1 by starting stimulation approximately 1 to 2 seconds after the trigger is detected. Since the timing when the capsule 1 reaches the root of the tongue, the pharynx or the like is different depending on the subject, the preset time from detection of the trigger to start of the stimulation may be made changeable depending on a person.

Moreover, the control unit 11 controls the stimulation unit 10 so as to end the stimulation after the stimulus is applied by the stimulation unit 10 for a predetermined period of time. Here, the period of time for which the stimulus is applied may be set in advance by a medical staff or the like or may be changed by the control unit 11 in accordance with the type of a disease, a degree of a symptom and the like.

The stimulation unit 10 has a function of applying a stimulus to a predetermined site in the body cavity. More specifically, the stimulation unit 10 can induce pharyngeal reflex and let the contents in the body be discharged by applying a stimulus to the root of the tongue or the pharynx. Moreover, the stimulation unit 10 can promote peristaltic movement and let the contents in the intestines be discharged by applying a stimulus to the colon.

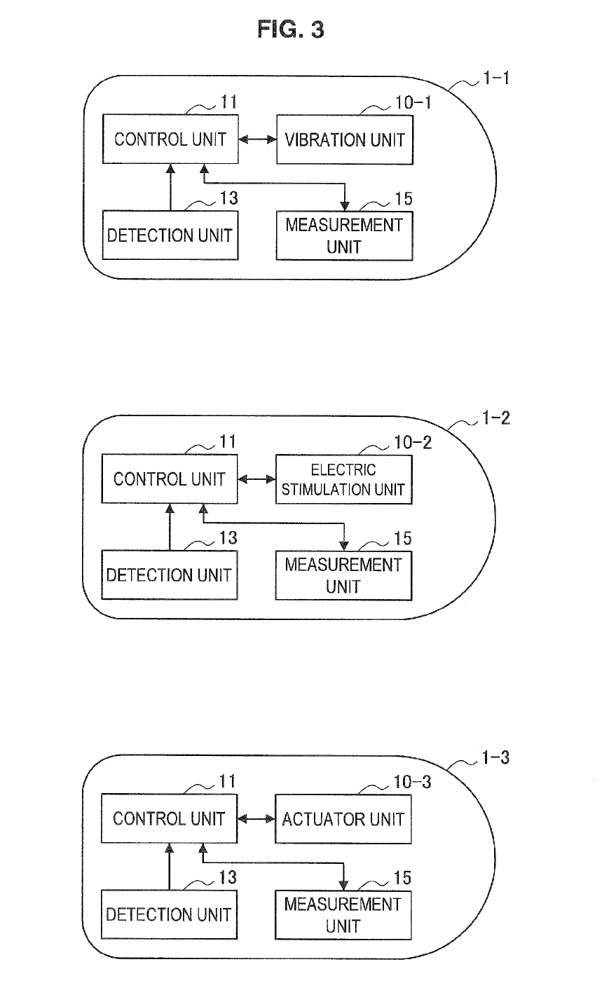

Here, the stimulus by the stimulation unit 10 may be more specifically vibration, electric stimulation or a pressure, for example. The specific stimulus will be explained below by referring to FIG. 3. FIG. 3 is a block diagram illustrating a configuration of the capsule according to a variation of this embodiment.

As illustrated in FIG. 3, a capsule 1-1 according to the variation of this embodiment has a vibration unit 10-1, the control unit 11, the detection unit 13, and the measurement unit 15. Since the control unit 11, the detection unit 13, and the measurement unit 15 are as explained by referring to FIG. 2, explanation here will be omitted.

A vibration unit 10-1 has a structure of vibrating by itself and can apply a stimulus to a predetermined site by vibrating. The vibration unit 10-1 may be a vibration motor formed of a coreless motor and an eccentric weight, for example.

Moreover, as illustrated in FIG. 3, a capsule 1-2 according to another variation in this embodiment has an electric stimulation unit 10-2, the control unit 11, the detection unit 13, and the measurement unit 15. Since the control unit 11, the detection unit 13, and the measurement unit 15 are as explained by referring to FIG. 2, explanation here will be omitted.

The electric stimulation unit 10-2 can apply a stimulus to a predetermined site by having an electric current flow. The electric stimulation unit 10-2 may have a structure of having an electric current flow to the predetermined site from one or a plurality of electrodes provided on an outer peripheral surface of the capsule 1-2, for example.

Moreover, as illustrated in FIG. 3, a capsule 1-3 according to another variation in this embodiment has an actuator unit 10-3, the control unit 11, the detection unit 13, and the measurement unit 15. Since the control unit 11, the detection unit 13, and the measurement unit 15 are as explained by referring to FIG. 2, explanation here will be omitted.

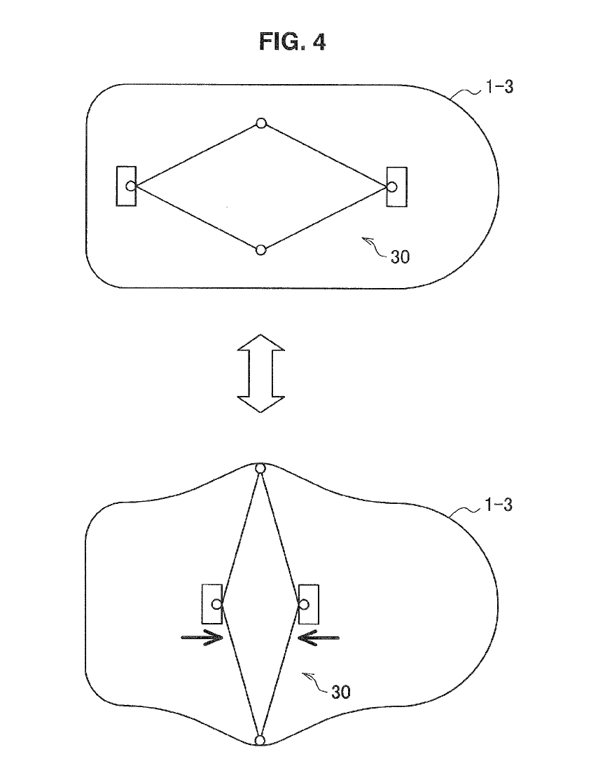

The actuator unit 10-3 can apply a stimulus to a predetermined site by driving the capsule 1-3 so as to be mechanically deformed. For example, the actuator unit 10-3 may be a solenoid driving so as to push open a pantograph structure 30 inside as illustrated in FIG. 4 or may be a DC motor driving so as to push open a jack (not shown) inside the capsule 1.

Moreover, the capsule 1-3 according to this variation is a deformable housing and is deformed by being pushed open by the actuator unit 10-3 from inside as illustrated in FIG. 4 so as to apply a stimulus to the predetermined site.

(1-3. Operation Processing)

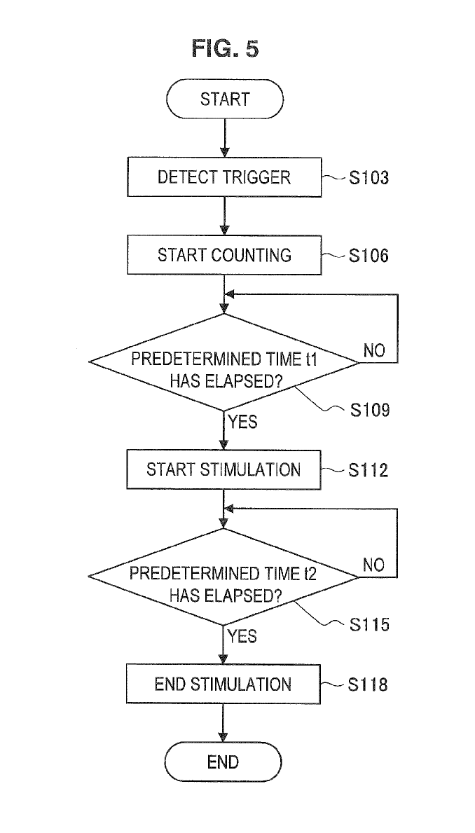

Subsequently, the operation processing of the capsule 1 according to this embodiment will be explained by referring to FIG. 5. FIG. 5 is a flowchart illustrating the operation processing of the capsule 1 according to the first embodiment. As illustrated in FIG. 5, first, the detection unit 13 detects a trigger (Step S103).

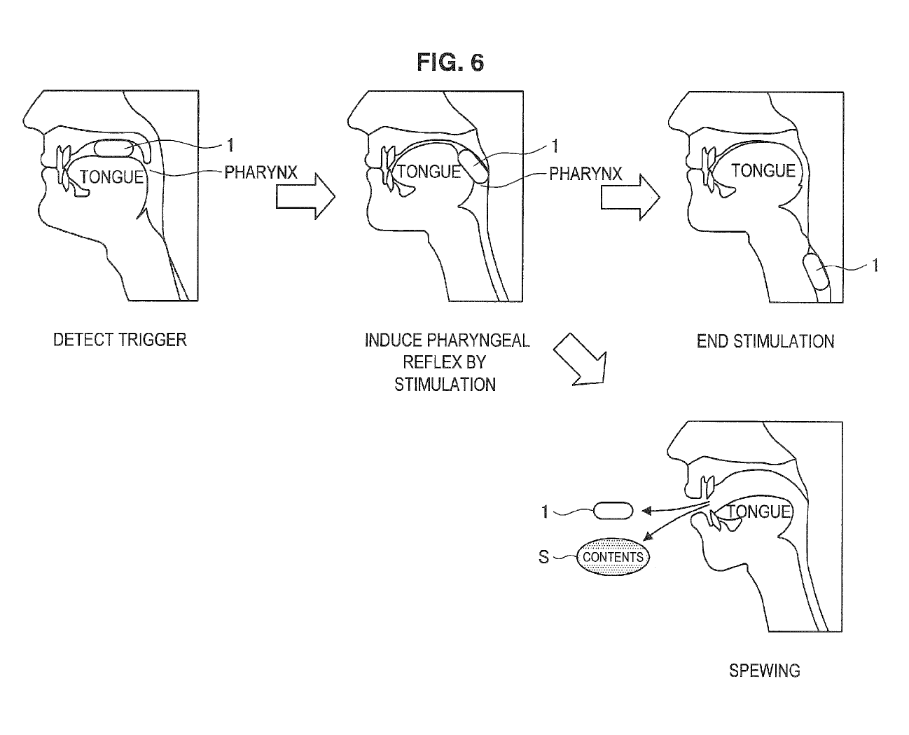

The trigger is detected when the switch is pushed down as described above or when a pressure is applied to the entire capsule 1 from outside. Thus, as illustrated in FIG. 6, for example, the capsule 1 may detect application of a pressure by the tongue or tooth in the mouth as a trigger when the capsule 1 is swallowed.

Subsequently, the measurement unit 15 starts counting (Step S106). Then, the control unit 11 starts stimulation (Step S112) to a predetermined site by controlling the stimulation unit 10 when a predetermined time t1 is counted by the measurement unit 15 (Step S109/Yes).

If the predetermined time t1 is approximately 1 to 2 seconds, for example, the capsule 1 can start stimulation at timing of reaching the vicinity of the pharynx as illustrated in FIG. 6. As a result, the capsule 1 induces pharyngeal reflex and is spewed together with the contents S in the body.

Subsequently, if a predetermined time t2 is counted by the measurement unit 15 from the start of stimulation (Step S115/Yes), the control unit 11 ends the stimulation to the predetermined site by controlling the stimulation unit 10 (Step S118). As a result, even if the pharynx is stimulated as illustrated in FIG. 6, for example, spewing is not caused, and even if the capsule 1 is swallowed into the body, the capsule 1 can be safely discharged without stimulating the other sites by controlling so that the stimulation is stopped after several seconds.

The capsule 1 according to the first embodiment of the present disclosure has been explained in detail. As described above, the capsule 1 according to this embodiment can apply a stimulus to induce pharyngeal relax to the predetermined site by starting stimulation after preset time has elapsed since the trigger is detected. The above described predetermined time t1 is set to time for the capsule 1 to reach the vicinity of the predetermined site. Since the time for the capsule 1 to reach the vicinity of the predetermined site might be different depending on a person, the predetermined time t1 may be changed depending on a subject. Moreover, the above described predetermined time t2 may be changed in accordance with the type of a disease, a degree of a symptom and the like.

### 2. SECOND EMBODIMENT

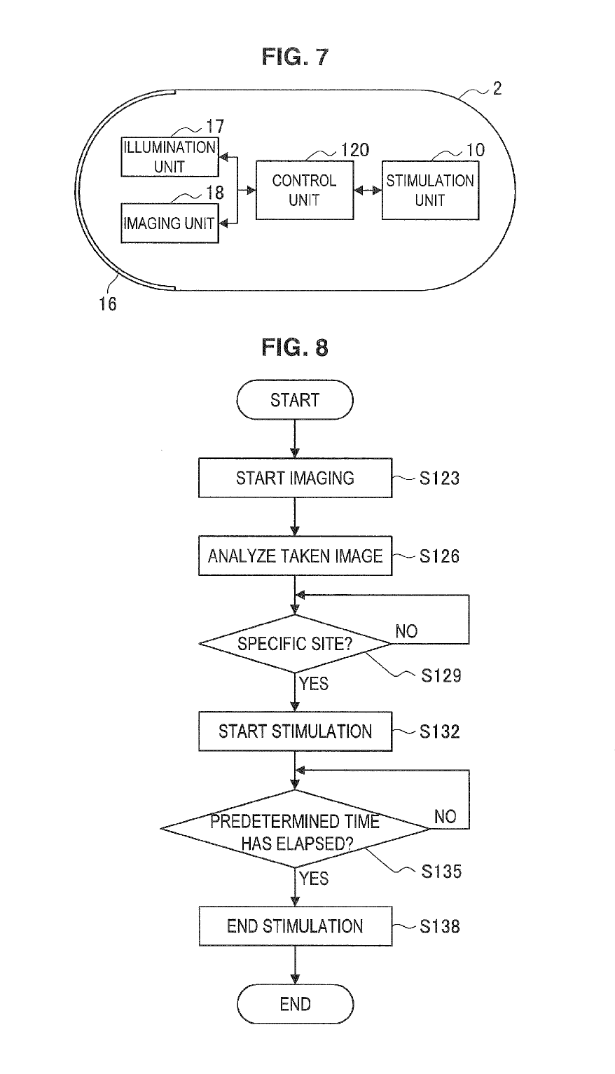

As described above, in the first embodiment, stimulation is started when the preset time has elapsed since the trigger is detected. However, the timing to start stimulation according to the present disclosure is not limited to the first embodiment, and stimulation may be started when it is determined that a specific site registered in advance is reached, for example. That will be explained below specifically by referring to FIGS. 7 to 8.

(2-1. Configuration of Capsule)

FIG. 7 is a block diagram illustrating a configuration of a capsule 2 according to a second embodiment. As illustrated in FIG. 7, the capsule 2 has the stimulation unit 10, a control unit 120, an illumination unit 17, and an imaging unit 18. Since the stimulation unit 10 is similar to that in the above-described first embodiment, explanation here will be omitted.

As illustrated in FIG. 7, the illumination unit 17 and the imaging unit 18 are arranged inside a transparent cover 16. The illumination unit 17 is realized by a while LED or the like and has a function of illuminating the inside of a body cavity. The imaging unit 18 is realized by an imaging optical system, an imaging sensor and the like and takes an image of the inside of the body cavity. Moreover, the imaging unit 18 outputs a taken image to the control unit 120.

The control unit 120 controls each configuration of the capsule 2. Specifically, the control unit 120 analyzes the taken image outputted from the imaging unit 18 and determines whether or not it is a specific site registered in advance. Moreover, if it is determined to be the specific site on the basis of the analysis of the taken image, the control unit 120 applies a stimulus to the specific site by controlling the stimulation unit 10.

For example, if a “colon” is registered as the specific site, the control unit 120 can apply a stimulus to the colon by controlling the stimulation unit 10 when it is determined to be the “colon” on the basis of the analysis of the taken image.

(2-2. Operation Processing)

Subsequently, operation processing of the capsule 2 according to this embodiment will be explained by referring to FIG. 8. FIG. 8 is a flowchart illustrating the operation processing of the capsule 2 according to the second embodiment. As illustrated in FIG. 8, first, power is turned on, and the capsule 2 swallowed by the subject 6 takes images while moving through the body cavity (Step S123).

Subsequently, the control unit 120 analyzes the taken image outputted from the imaging unit 18 (Step S126). Subsequently, when it is determined to be the specific site registered in advance as the result of analysis of the taken image (Step S129/Yes), the control unit 120 starts stimulation to the specific site by controlling the stimulation unit 10 (Step S132).

Registration of the specific site is made by the subject 6 or a medical staff in advance by using an information processing device (not shown), for example, and information of the specific site registered in the information processing device as above may be stored in the capsule 2.

Subsequently, if predetermined period of time has elapsed since start of the stimulation (Step S135/Yes), the control unit 120 ends stimulation to the specific site by controlling the stimulation unit 10 (Step S138). For example, the capsule 2 can promote peristaltic movement more effectively by continuing stimulation for the predetermined period of time while advancing through the colon. Then, the capsule 2 is discharged safely without stimulating the other sites.

The capsule 2 according to the second embodiment of the present disclosure has been explained in detail. As described above, according to this embodiment, a stimulus can be applied to the specific site registered in advance. Moreover, by applying a stimulus to the colon, for example, peristaltic movement is promoted, and a foreign substance in the intestines can be discharged. Determination on whether or not it is the specific site according to the second embodiment can be made not only by analysis of the taken image but also on the basis of values detected by various sensors, for example.

### 3. THIRD EMBODIMENT

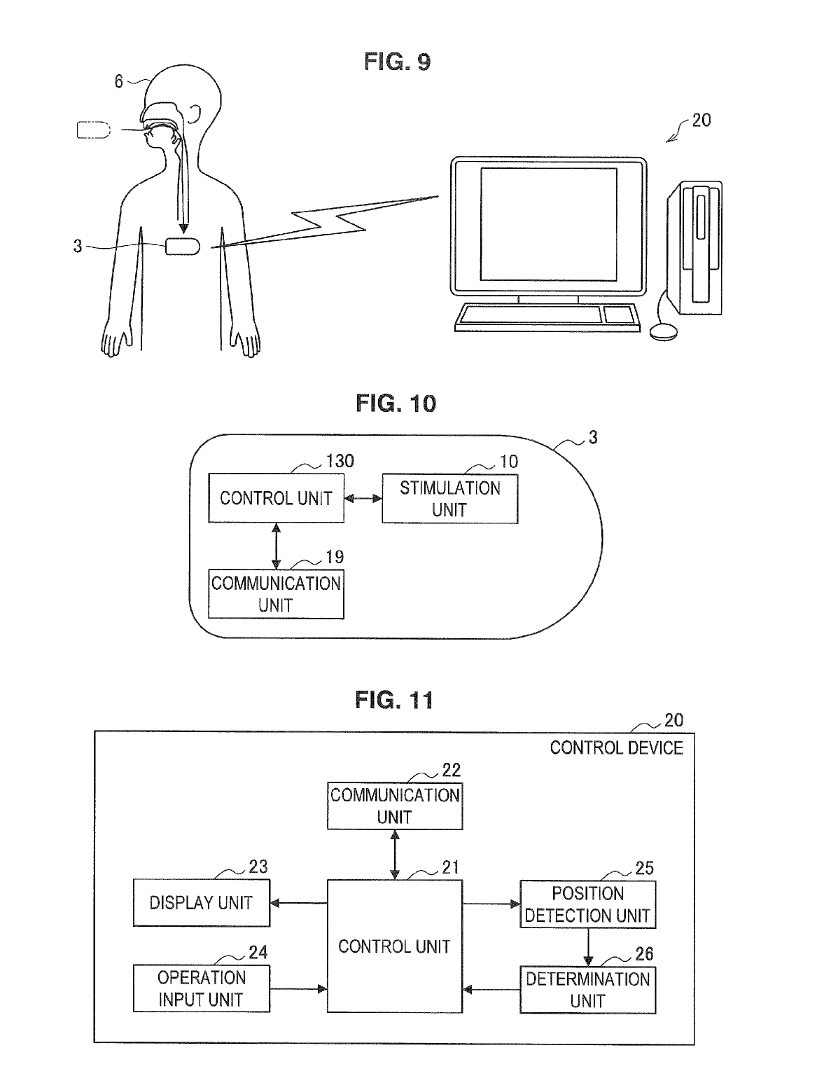

As described above, in the first and second embodiments, the capsule-type medical device determines timing to start stimulation but the timing to start stimulation according to the present disclosure is not limited to that and stimulation may be started in accordance with a control signal from an external device, for example. A capsule-type medical device according to a third embodiment which starts stimulation in accordance with a control signal from the external device will be explained below specifically by referring to FIGS. 9 to 12.

(3-1. Entire Configuration)

FIG. 9 is an explanatory view illustrating an entire configuration of the third embodiment. As illustrated in FIG. 9, a capsule-type medical device 3 (hereinafter referred to also as a capsule 3) according to this embodiment conducts data communication with an external control device 20 and can promote discharge of contents by applying a stimulus to a predetermined site in the body.

The capsule 3 and the control device 20 may conduct direct data communication or may conduct data communication via another device.

(3-2. Each Configuration)

(Configuration of Capsule 3)

FIG. 10 is a block diagram illustrating a configuration of the capsule 3 according to the third embodiment. As illustrated in FIG. 10, the capsule 3 has the stimulation unit 10, a control unit 130, and a communication unit 19. Since the stimulation unit 10 is similar to that in the above described first embodiment, explanation here will be omitted.

The control unit 130 controls each configuration of the capsule 3. More specifically, the control unit 130 according to this embodiment receives a control signal received by the communication unit 19 from the control device 20 and controls the stimulation unit 10 so that the stimulation to a predetermined site is started or ended in accordance with the control signal.

The communication unit 19 conducts transmission/reception of data with the control device 20. More specifically, the communication unit 19 according to this embodiment transmits positional information which is a signal for position detection to the control device 20 and receives a control signal from the control device 20.

(Configuration of Control Device 20)

FIG. 11 is a block diagram illustrating a configuration of the control device 20 according to the third embodiment. As illustrated in FIG. 11, the control device 20 has a control unit 22, a communication unit 22, a display unit 23, an operation input unit 24, a position detection unit 25, and a determination unit 26.

The communication unit 22 has a function of connecting to an external device and conducting transmission/reception of data. For example, the communication unit 22 according to this embodiment conducts data transmission/reception with the capsule 3. Specifically, the communication unit 22 receives positional information from the capsule 3 and the communication unit 22 may also transmit a control signal to the capsule 3.

The display unit 23 has a function of performing screen display including an image and a text in accordance with control of the control unit 21. The display unit 23 may be realized by a liquid crystal display (LCD), an organic light-emitting diode (OLED) or a cathode ray tube (CRT) and the like.

The operation input unit 24 has a function of detecting an operation by a user (medical staff, the subject 6 or the like) and outputting an input signal generated on the basis of the detected operation input to the control unit 21. Moreover, the operation input unit 24 is realized by a mouse, a keyboard, a touch panel and the like.

The position detection unit 25 detects (calculates) a position of the capsule 3 on the basis of the positional information which is a signal for position detection received by the communication unit 22 from the capsule 3. Then, the position detection unit 25 outputs the detected position of the capsule 3 to the determination unit 26.

The position detection unit 25 may detect (calculate) the position of the capsule 3 by analyzing a taken image received from the capsule 3 in addition to the signal for position detection. Moreover, the position detection unit 25 may detect (calculate) the position of the capsule 3 on the basis of the values (pH value and the like) detected by various sensors received from the capsule 3.

The determination unit 26 determines whether or not the position of the capsule 3 detected by the position detection unit 25 is in the vicinity of a specific site registered in advance by the medical staff, the subject 6 or the like and outputs the determination result to the control unit 21.

The control unit 21 has a function of controlling each configuration of the control device 20. For example, the control unit 21 according to this embodiment controls the communication unit 22 so that the control signal is transmitted to the capsule 3 on the basis of the determination result outputted from the determination unit 26.

More specifically, the control unit 21 transmits a control signal (start signal) for starting stimulation to the capsule 3 when it is determined by the determination unit 26 that the capsule 3 has reached the vicinity of the predetermined site. Moreover, the control unit 21 may transmit a control signal (end signal) for ending the stimulation to the capsule 3 when a predetermined period of time has elapsed since the start signal is transmitted to the capsule 3.

(3-3. Operation Processing)

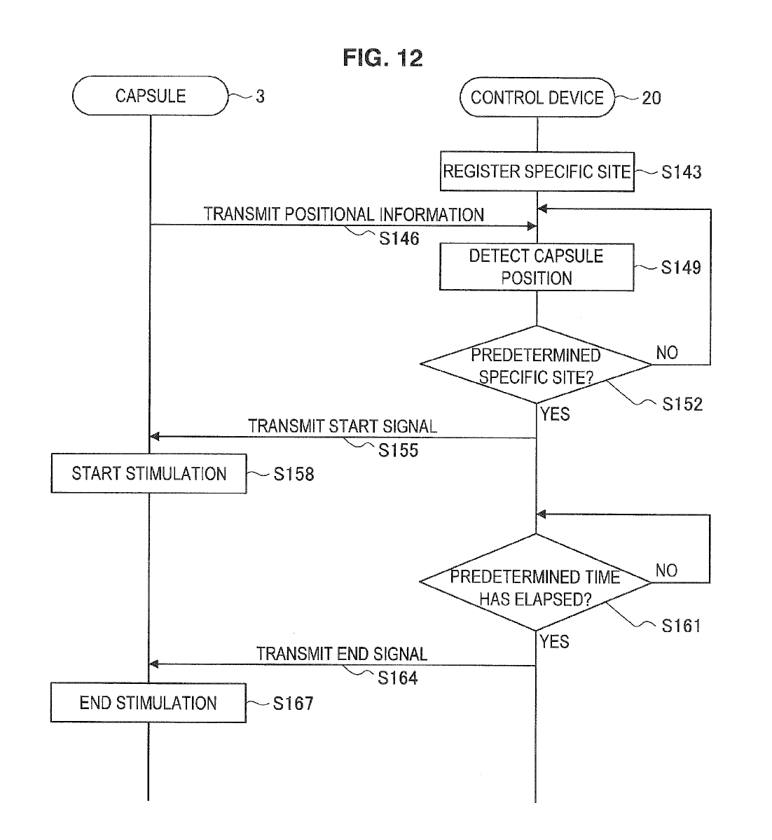

Subsequently, operation processing of a medical system according to this embodiment will be explained by referring to FIG. 12. FIG. 12 is a flowchart illustrating the operation processing of the medical system according to the third embodiment. As illustrated in FIG. 12, first, a specific site is registered in the control device 20 (Step S143). In registration of the specific site, a predetermined site in a body cavity to be stimulated is specified by a medical staff, the subject 6 or the like. Moreover, in the registration of the specific site, the control device 20 may receive specification of stimulation time and specification of a capsule according to the type of stimulation (vibration capsule, electric stimulation capsule, deformation capsule or the like).

Subsequently, power is turned on, and the capsule 3 having been swallowed by the subject 6 transmits positional information to the control device 20 while moving through the body cavity (Step S146).

Subsequently, the position detection unit 25 of the control device 20 detects a position of the capsule 3 on the basis of intensity of the positional information (signal for position detection) transmitted from the capsule 3 (Step S149). If a taken image in the body cavity is transmitted from the capsule 3, the position detection unit 25 may detect a position of the capsule 3 by analyzing the taken image.

Then, the determination unit 26 of the control device 20 determines whether or not the position of the capsule 3 detected by the position detection unit 25 is in the vicinity of the specific site registered at the above described Step S143 (Step S152). For example, if the capsule 3 is within a predetermined distance from the specific site registered in advance, the determination unit 26 may determine that the capsule 3 has reached the vicinity of the specific site.

Subsequently, if it is determined by the determination unit 26 that the position of the capsule 3 is in the vicinity of the specific site, the control device 20 transmits a control signal for starting stimulation (start signal) to the capsule 3 (Step S155).

Then, the control unit 130 of the capsule 3 controls the stimulation unit 10 so that stimulation to the specific site is started in accordance with the start signal received from the control device 20 (Step S158).

Subsequently, the control unit 21 of the control device 20 determines whether or not predetermined period of time has elapsed since the start signal is transmitted (Step S161). Here, the predetermined period of time may be time set in advance or may be time specified at the same time as the registration of the specific site at the above described Step S143.

Then, after the predetermined time has elapsed, the control unit 21 of the control device 20 transmits a control signal (end signal) for ending the stimulation to the capsule 3 (Step S164).

Then, the control unit 130 of the capsule 3 controls the stimulation unit 10 so that the stimulation to the specific site is ended in accordance with the end signal received from the control device 20 (Step S167).

The capsule 3 according to the third embodiment of the present disclosure is explained in detail as above. As described above, according to this embodiment, a stimulus can be applied for a predetermined period of time to a specific site registered in advance.

As a result, the capsule 3 according to this embodiment can promote peristaltic motion more effectively and can cause foreign substances in the intestines to be discharged by applying a stimulus for a predetermined period of time while advancing in the colon, for example.

Moreover, the capsule 3 is discharged safely without stimulating the other sites since the stimulus to the specific site is ended in accordance with the end signal from the control device 20.

### 4. CONCLUSION

As described above, the capsule-type medical device according to the embodiments of the present disclosure can cause contents to be discharged from inside the body safely and casually by applying a stimulus to a predetermined site in the body cavity. As a result, a foreign substance swallowed by mistake, for example, can be discharged safely from inside the body.

More specifically, the capsule-type medical device according to the embodiments of the present disclosure can induce pharyngeal reflex and cause the contents to be spewed by applying a stimulus to the root of the tongue or to the vicinity of the pharynx. Here, since the capsule 1 according to the first embodiment starts stimulation to the predetermined site after the predetermined period of time has elapsed since the trigger is detected, the stimulus can be applied to the vicinity of the pharynx by detecting a trigger when being swallowed, for example, and by starting stimulation approximately after 1 to 2 seconds.

Moreover, the capsule-type medical device according to the embodiments of the present disclosure can promote peristaltic movement and cause the contents in the intestines to be discharged by applying a stimulus into the colon. Here, the capsules 2 and 3 in the above described second and third embodiments start stimulation to the specific site when it is determined by the capsule 2 or the control device 20 that the contents are in the vicinity of the specific site registered in advance. Thus, by registering the colon as the vicinity of the specific site in the above described second and third embodiments, for example, the capsules 2 and 3 can apply a stimulus into the colon and promote peristaltic movement.

The preferred embodiments of the present disclosure have been described above with reference to the accompanying drawings, whilst the present technology is not limited to the above examples, of course. A person skilled in the art may find various alterations and modifications within the scope of the appended claims, and it should be understood that they will naturally come under the technical scope of the present disclosure.

Additionally, the present technology may also be configured as below.

(1)

A capsule-type medical device including:

a stimulation unit configured to apply a stimulus to a predetermined site in a body cavity.

(2)

The capsule-type medical device according to (1), wherein

the stimulus applied by the stimulation unit is vibration, electric stimulation or a pressure.

(3)

The capsule-type medical device according to (1) or (2), wherein

the stimulation unit starts stimulation to the predetermined site after a period of time set in advance has elapsed since detecting a trigger and ends the stimulation after a certain period of time has elapsed since starting the stimulation.

(4)

The capsule-type medical device according to (3), wherein

the stimulation unit starts stimulation to a throat approximately 1 to 2 seconds after detecting application of a pressure.

(5)

The capsule-type medical device according to (1) or (2), further including:

an imaging unit configured to take an image of an inside of the body cavity,

wherein, when a site is determined to be a specific site on the basis of a taken image taken by the imaging unit, the stimulation unit starts the stimulation to the specific site.

(6)

The capsule-type medical device according to (1) or (2), further including:

a receiving unit configured to receive a control signal form an external device,

wherein the stimulation unit starts or ends the stimulation to the site in accordance with a control signal received from the external device.

(7)

A system including:

a capsule-type medical device which has a stimulation unit configured to apply a stimulus to a predetermined site in a body cavity; and

an external device configured to transmit a control signal for controlling start or end of the stimulation,

wherein the capsule-type medical device further has a receiving unit configured to receive the control signal from the external device, and

wherein the stimulation unit starts or ends the stimulation to the predetermined site in accordance with the control signal received from the external device.

### REFERENCE SIGNS LIST

1, 1-1, 1-2, 1-3, 2, 3 capsule-type medical device
6 subject
10 stimulation unit
10-1 vibration unit
10-2 electric stimulation unit
10-3 actuator unit
11, 120, 130 control unit
13 detection unit
15 measurement unit
16 transparent cover
17 illumination unit
18 imaging unit
20 control device
21 control unit
22 communication unit
23 display unit
24 operation input unit
25 position detection unit
26 determination unit
30 pantograph structure

1. A capsule-type medical device comprising:
a stimulation unit configured to apply a stimulus to a predetermined site in a body cavity.

2. The capsule-type medical device according to claim 1, wherein
the stimulus applied by the stimulation unit is vibration, electric stimulation or a pressure.

3. The capsule-type medical device according to claim 1, wherein
the stimulation unit starts stimulation to the predetermined site after a period of time set in advance has elapsed since detecting a trigger and ends the stimulation after a certain period of time has elapsed since starting the stimulation.

4. The capsule-type medical device according to claim 3, wherein
the stimulation unit starts stimulation to a throat approximately 1 to 2 seconds after detecting application of a pressure.

5. The capsule-type medical device according to claim 1, further comprising:
an imaging unit configured to take an image of an inside of the body cavity,
wherein, when a site is determined to be a specific site on the basis of a taken image taken by the imaging unit, the stimulation unit starts the stimulation to the specific site.

6. The capsule-type medical device according to claim 1, further comprising:
a receiving unit configured to receive a control signal form an external device,
wherein the stimulation unit starts or ends the stimulation to the site in accordance with a control signal received from the external device.

7. A system comprising:
a capsule-type medical device which has a stimulation unit configured to apply a stimulus to a predetermined site in a body cavity; and
an external device configured to transmit a control signal for controlling start or end of the stimulation,
wherein the capsule-type medical device further has a receiving unit configured to receive the control signal from the external device, and
wherein the stimulation unit starts or ends the stimulation to the predetermined site in accordance with the control signal received from the external device.
